(19) United States
(12) Patent Application Publication
(10) Pub. No.: US 2018/0104460 A1
(43) Pub. Date: Apr. 19, 2018

(54) DYNAMIC STRETCHING TOOLS AND METHODS

(71) Applicant: Christin Diane Peters, Redwood City, CA (US)

(72) Inventor: Christin Diane Peters, Redwood City, CA (US)

(21) Appl. No.: 15/294,764

(22) Filed: Oct. 16, 2016

(51) Int. Cl. A61M 29/00; A61H 19/00

(52) U.S. Cl. CPC A61M 29/00; A61H 2205/087; A61H 19/44; A61H 19/00

(57) ABSTRACT

Therapeutic tools and methods for treating vaginal pain experienced during penetration.

### PRIORITY OF INVENTION

Not applicable.

### FIELD

The present invention relates to the field of therapeutic tools. More particularly, the present invention relates to a therapeutic tool for women's health. In some embodiments, the invention relates to a therapeutic tool for a women's health condition involving vaginal pain experienced during penetration.

### BACKGROUND

Women suffering from vaginal pain experienced during penetration are treated by professionals in a wide variety of fields, including oncology, radiotherapy, gynecology, obstetrics and physical therapy. Such pain can be a result of many causes, including Anorgasmia, Chronic Pelvic Pain, Dyspareunia, Levator Ani Spasm, Neuropathic Pain, Painful Scar Tissue, Tight Vaginal Opening, Vaginismus, and Vulvodynia. Current therapeutic methods for treating vaginal pain experienced during penetration involving using of a dilator or similar tool. A dilator is a tool for statically stretching a vagina by inserting a dilator into the vagina such that the dilator's diameter causes slight discomfort by stretching the vagina. Continued therapy using a series of dilators with increasing diameters stretch the vagina over time and reduce or eliminate pain experienced during penetration.

Alternatively, a woman may use her own thumb to dynamically stretch her vagina by inserting her thumb into her vagina and rotating her wrist. Dynamic stretching is a preferred therapy over static stretching, such as the dilator, for treating pain relating to activities involving movement, however a prescribed therapy for a woman experiencing vaginal pain during penetration can require up to over a minute of continuous dynamic stretching which places the patient at risk of developing tendonitis or thumb pain due to the repetitive stress placed on the patient's thumb. Additionally, some patients experience general discomfort with positioning their thumb, which can lead to noncompliance with prescribed home therapies and prolong the need for medical services.

Thus, there remains a need for a tool that provides a woman experiencing vaginal pain during penetration with a means of dynamically stretching her vagina without risk of tendonitis, thumb pains, or general discomfort. Pelvic floor massage tools, such as those described in United States Design Pat. Nos. D712,057 and D720,862 and marketed products such as Therawand®, are attempts to remedy this need, however, these tools are limited by an S-shape that is not optimal for prescribed prolonged therapeutic methods. Moreover, these tools lack an ability to provide a variety attachments, such as a dilator, having different diameters or shapes. The present invention remedies these deficiencies and more.

### SUMMARY

The present invention provides a tool possessing the utility of allowing a woman to dynamically stretch her vagina.

In some embodiment, the invention provides a therapeutic tool comprising a long component, a U-bend component, and a short component wherein:

the long component is at least 30 cm (˜12 in.).

In some embodiments, the invention provides a therapeutic tool comprising a long component, a U-bend component, and a short component wherein:

the long component is retractable and when extended is at least 30 cm (˜12 in.).

In some embodiments, the invention provides a method of treating a human female having vaginal pain during vaginal penetration comprising a therapeutic tool comprising a long component, a U-bend component, and a short component wherein:

the long component is at least 30 cm (˜12 in.).

In some embodiments, the invention provides a method of treating a human female having vaginal pain during vaginal penetration comprising a therapeutic tool comprising a long component, a U-bend component, and a short component wherein:

the long component is retractable and when extended is at least 30 cm (˜12 in.).

### BRIEF DESCRIPTION OF THE DRAWINGS

Figures are provided to assist in the understanding of the claimed subject matter. One having ordinary skill in the art would understand that the Figures depict specific non-limiting embodiments of the claimed invention, and are not included to represent the all embodiments of the invention. Figures are not drawn to scale.

FIGS. 1-6 show varying perspectives of embodiments of the invention.

FIG. 1 shows a side perspective of embodiments of the invention comprising a long component, a U-bend component, and a short component, wherein the long component is attached or affixed to the U-bend component at one end of the U-bend component and the short component is attached or affixed to the U-bend component on the other end of the U-bend component.

FIG. 2 shows a side perspective of embodiments of the invention comprising a single piece comprising a long component, a U-bend component, and a short component.

FIG. 3 shows a side perspective of embodiments of the invention comprising a long component, a U-bend component, and a short component, wherein the short component is optionally attached with a tip; and the tip is a vaginal dilator.

FIG. 4 shows a side perspective of embodiments of the invention comprising a long component, a U-bend component, and a short component, wherein the short component is optionally attached with a tip; and the tip is a vaginal dilator.

FIG. 5 shows a side perspective of embodiments of the invention comprising a long component, a U-bend component, and a short component, wherein the long component is optionally attached with a handle.

FIG. 6 shows a side perspective of embodiments of the invention comprising a long component, a U-bend component, and a short component, wherein the long component is optionally attached with a handle; the short component is optionally attached with a tip; and the tip is a vaginal dilator.

### DESCRIPTION

Reference will now be made in detail to certain claims of the invention, examples of which are illustrated in the accompanying figures. While the invention will be described in conjunction with the enumerated claims, it will be understood that they are not intended to limit the invention to those claims. On the contrary, the invention is intended to cover all alternatives, modifications, and equivalents, which may be included within the scope of the present invention as defined by the claims. For example, where a particular feature is disclosed in the context of a particular aspect or embodiment of the invention, or a particular claim, that feature can also be used, to the extent possible, in combination with and/or in the context of other particular aspect and embodiments of the invention, and in the invention generally.

Where reference herein to a method comprising two or more defined steps, the defined steps can be carried in any order or simultaneously (except where the context excludes that possibility), and the method can include one or more other steps which are carried out before any of the defined steps, between two of the defined steps, or after all of the defined step (except where the context excludes that possibility).

### Definitions

Unless stated otherwise, the following terms and phrases as used herein are intended to have the following meanings:

“Component” refers to subpart of the invention. Components are not required to be distinct or separate. In some embodiments, the invention is a single piece comprising multiple components. In some embodiments, the invention is multiple pieces comprising multiple components.

“Comprises” and grammatical equivalents thereof are used herein to mean that other components are optionally present. For example, an article “comprising” components A, B, and C, can consist of components A, B, and C or can contain not only components A, B, and C, but also one or more other components.

“Dilator” refers to a therapeutic instrument for enlarging or stretching the vagina. Non-limiting examples of dilator shapes include cylindrical and hemispherical.

“Penetration” refers to any breaching of the vaginal introitus. Non-limiting examples of penetration include breaching a woman's vaginal introitus using a finger, a speculum, tampon, penis, dilator, or vibrator.

“Retractable” refers to a component that may exist in multiple lengths. For example, some embodiments include a long component that can be 30 cm (˜12 in.), 33 cm (˜13 in.), 36 cm (˜14 in.) . . . 84 cm (˜33 in.), 87 cm (˜34 in.), 90 cm (˜35 in.) depending on the user's preference.

“Therapeutic” refers to treatment of a patient in need thereof wherein the patient has a medical condition. Non-limited examples of medical conditions include Anorgasmia, Chronic Pelvic Pain, Dyspareunia, Levator Ani Spasm, Neuropathic Pain, Painful Scar Tissue, Tight Vaginal Opening, Vaginismus, and Vulvodynia.

“Tip” refers to an optional subpart of the invention that may be attached to the short component. The tip may be attached or affixed by a variety of means. Non-limiting examples of means of attaching of affixing a tip to the short component include a threaded screw and clip-on.

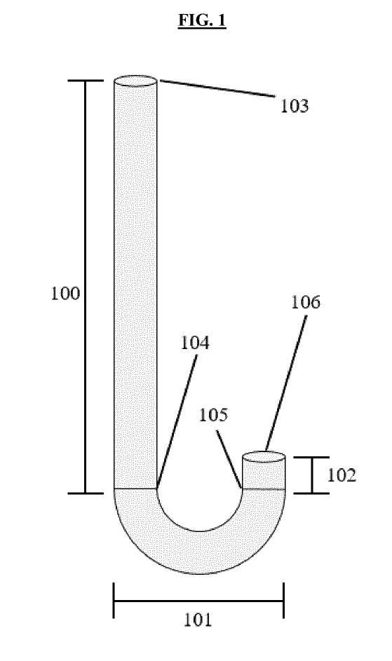

‘U-bend” refers to a half-oval shape. A U-bend can be symmetrical or asymmetrical. A U-bend's shape can be similar to the letter “U,” multiple straight lines such as three straight lines “ [symbol] ,” or five straight lines “ [symbol] ” or more straight lines. “Ends” of a U-bend refer to the two prongs. 104 and 105 of FIG. 1 provide specific non-limiting embodiments of ends of a U-bend component.

### DETAILED DESCRIPTION

Specific depictions as well as specific embodiments of the invention described herein are for illustration only; they do not exclude other configurations.

Therapeutic Tools

The present invention provides a tool possessing the utility of allowing a woman to dynamically stretch her vagina. Dynamically stretching a muscle can reduce the duration of therapy and better mimic active movements. A therapeutic tool for actively stretching is provided.

In some embodiments, the invention provides a therapeutic tool comprising a long component, a U-bend component, and a short component wherein:

the long component is at least 30 cm (˜12 in.).

In some embodiments, the invention provides a therapeutic tool comprising a long component, a U-bend component, and a short component wherein:

the long component is at least 30 cm (˜12 in.); and

the short component is optionally attached with a tip.

In some embodiments, the invention provides a therapeutic tool comprising a long component, a U-bend component, and a short component wherein:

the long component is at least 30 cm (˜12 in.);

the short component is optionally attached with a tip; and
the tip is a vaginal dilator.

In some embodiments, the invention provides a therapeutic tool comprising a long component, a U-bend component, a short component wherein:

the long component is at least 30 cm (˜12 in.);

the long component is attached to one end of the U-bend component;

the short component is attached to the other end of the U-bend component;

the short component is optionally attached with a tip; and
the tip is a vaginal dilator.

In some embodiments, the invention provides a therapeutic tool comprising a long component, a U-bend component, and a short component wherein:

the long component is between 30 cm (˜12 in.) and 90 cm (˜35 in.).

In some embodiments, the invention provides a therapeutic tool comprising a long component, a U-bend component, and a short component wherein:

the long component is between 30 cm (˜12 in.) and 90 cm (˜35 in.).; and

the short component is optionally attached with a tip.

In some embodiments, the invention provides a therapeutic tool comprising a long component, a U-bend component, and a short component wherein:

the long component is between 30 cm (˜12 in.) and 90 cm (˜35 in.);

the short component is optionally attached with a tip; and
the tip is a vaginal dilator.

In some embodiments, the invention provides a therapeutic tool comprising a long component attached, a U-bend component, and a short component wherein:

the long component is between 30 cm (˜12 in.) and 90 cm (˜35 in.);

the long component is attached to one end of the U-bend component;

the short component is attached to the other end of the U-bend component;

the short component is optionally attached with a tip; and
the tip is a vaginal dilator.

In some embodiments, the invention provides a therapeutic tool comprising a long component, a U-bend component, and a short component wherein:

the long component is retractable and when extended is between 30 cm (˜12 in.) and 90 cm (˜35 in.).

In some embodiments, the invention provides a therapeutic tool comprising a long component, a U-bend component, and a short component wherein:

the long component is retractable and when extended is between 30 cm (˜12 in.) and 90 cm (˜35 in.).; and

the short component is optionally attached with a tip.

In some embodiments, the invention provides a therapeutic tool comprising a long component, a U-bend component, and a short component wherein:

the long component is retractable and when extended is between 30 cm (˜12 in.) and 90 cm (˜35 in.);

the short component is optionally attached with a tip; and
the tip is a vaginal dilator.

In some embodiments, the invention provides a therapeutic tool comprising a long component, a U-bend component, and a short component wherein:

the long component is retractable and when extended is between 30 cm (˜12 in.) and 90 cm (˜35 in.);

the long component is attached to one end of the U-bend component;

the short component is attached to the other end of the U-bend component;

the short component is optionally attached with a tip; and
the tip is a vaginal dilator.

Where specific reference herein to a long component's measurement is provided, the measurement is respective to vertical measurement. Where specific reference herein to a short component's measurement is provided, the measurement is respective to vertical measurement. For example, a long component that is at least 30 cm (˜12 in.) refers to a long component having 30 cm (˜12 in.) between each end. For further example, a long component that is at least 30 cm (˜12 in.) refers to a long component having three dimensions: width X, length Y and height Z, wherein width X, length Y and height Z have a relationship are shown in the following plane:

wherein height Z would be a long component that is 30 cm (˜12 in.); and width X and length Y would be a measurement less than 30 cm (˜12 in.). In some embodiments, width X and length Y would independently be less than 10 cm (˜4 in.). In some embodiments, width X and length Y would independently be less than 5 cm (˜2 in.).

In some embodiments, the invention provides a long component. In some embodiments, the long component is at least 30 cm (˜12 in.). In some embodiments, the long component is between 30 cm (˜12 in.) and 90 cm (˜35 in.). In some embodiments, the long component is retractable. In some embodiments, the long component is retractable and when extended is between 30 cm (˜12 in.) and 90 cm (˜35 in.). In some embodiments, the long component is retractable and when retracted is between 18 cm (˜7 in.) and 23 cm (˜9 in.) and when extended is between 30 cm (˜12 in.) and 90 cm (˜35 in.).

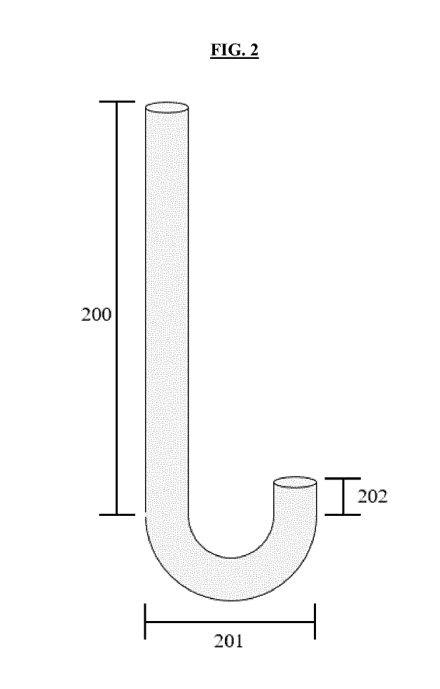

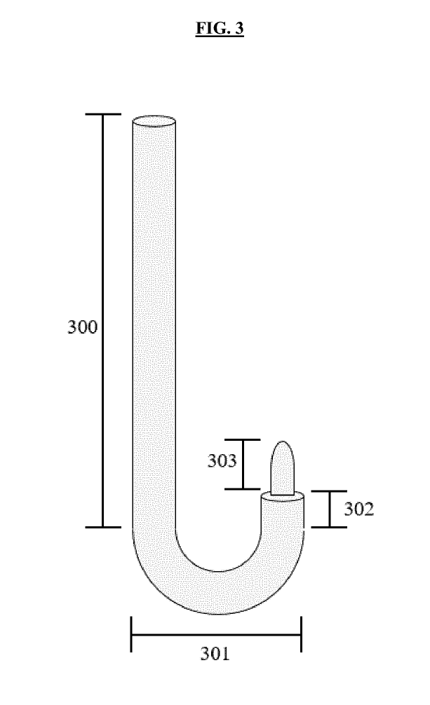

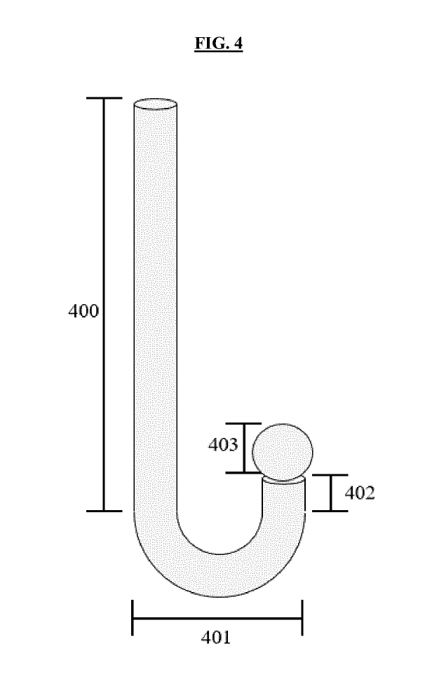

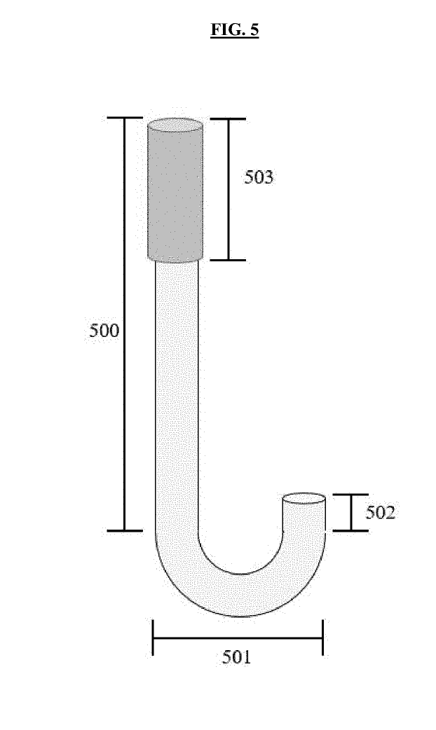

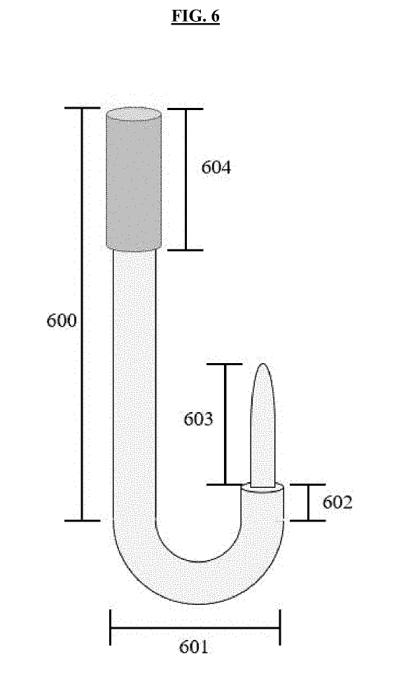

In some embodiments, the invention provides a long component as described in any one of FIG. 1, FIG. 2, FIG. 3, FIG. 4, FIG. 5, and/or FIG. 6. In some embodiments, the invention provides a long component as described in FIG. 1. In some embodiments, the invention provides a long component as described in FIG. 2. In some embodiments, the invention provides a long component as described in FIG. 3. In some embodiments, the invention provides a long component as described in FIG. 4. In some embodiments, the invention provides a long component as described in FIG. 5. In some embodiments, the invention provides a long component as described in FIG. 6.

In some embodiments, the invention provides a long component comprising a handle, wherein the handle is described in any one of FIG. 5, and/or FIG. 6. In some embodiments, the invention provides a long component comprising a handle as described in FIG. 5. In some embodiments, the invention provides a long component comprising a handle as described in FIG. 6.

In some embodiments, the invention provides a U-bend component. In some embodiments, the U-bend component is between the long component and the short component. In some embodiments, the U-bend component is symmetrical. In some embodiments, the U-bend component is asymmetrical. In some embodiments, the ends of the U-bend component are between 2.5 cm (˜1 in.) and 15 cm (˜6 in.). In some embodiments, the U-bend component is symmetrical wherein each half of the U-bend component is between 2.5 cm (˜1 in.) and 15 cm (˜6 in.). In some embodiments, the U-bend component is asymmetrical wherein each half of the U-bend component is between 2.5 cm (˜1 in.) and 15 cm (˜6 in.).

In some embodiments, the invention provides a U-bend component as described in any one of FIG. 1, FIG. 2, FIG. 3, FIG. 4, FIG. 5, and/or FIG. 6. In some embodiments, the invention provides a U-bend component as described in FIG. 1. In some embodiments, the invention provides a U-bend component as described in FIG. 2. In some embodiments, the invention provides a U-bend component as described in FIG. 3. In some embodiments, the invention provides a U-bend component as described in FIG. 4. In some embodiments, the invention provides a U-bend component as described in FIG. 5. In some embodiments, the invention provides a U-bend component as described in FIG. 6.

In some embodiments, the invention provides a short component. In some embodiments, the short component has a length of less than 15 cm (˜6 in.). In some embodiments, the short component has a length of less than 7.5 cm (˜3 in.). In some embodiments, the short component comprises a tip.

In some embodiments, the invention provides a short component as described in any one of FIG. 1, FIG. 2, FIG. 3, FIG. 4, FIG. 5, and/or FIG. 6. In some embodiments, the invention provides a short component as described in FIG. 1. In some embodiments, the invention provides a short component as described in FIG. 2. In some embodiments, the invention provides a short component as described in FIG. 3. In some embodiments, the invention provides a short component as described in FIG. 4. In some embodiments, the invention provides a short component as described in FIG. 5. In some embodiments, the invention provides a short component as described in FIG. 6.

In some embodiments, the invention provides a long component, a U-bend component and a short component wherein the long component, the U-bend component, and the short component are cylindrical.

In some embodiments, the invention provides a short component comprising a tip. In some embodiments, the tip is attached to the short component. In some embodiments, the tip is separate from the short component and can be attached or affixed to the short component. In some embodiments, the tip can be screwed on to the short component. In some embodiments, the tip can be clipped on to the short component. In some embodiments, the tip is a vaginal dilator. In some embodiments, the tip can be selected from a group of dilators with different diameters. In some embodiments, the tip can be selected from a group of dilators with different diameters wherein the diameters range from 13 mm (˜0.5 in.) to 151 mm (˜6 in.). In some embodiments, the tip can be selected from a group of dilators with different diameters wherein the diameters range from 13 mm (˜0.5 in.) to 44 mm (˜1.75 in.). In some embodiments, the tip can be selected from a group of dilators with different diameters wherein the diameters are selected from 13 mm (˜0.5 in.), 18 mm (˜0.70 in), 22 mm (˜0.86 in), 25 mm (˜1 in.), 29 mm (˜1.15 in), 32 mm (˜1.25 in.), 35 mm (˜1.37 in.), 39 mm (˜1.53 in.) and 44 mm (˜1.75 in.).

In some embodiments, the invention provides a short component comprising a tip, wherein the tip is described in any one of FIG. 3, FIG. 4, and/or FIG. 6. In some embodiments, the invention provides a short component comprising a tip as described in FIG. 3. In some embodiments, the invention provides a short component comprising a tip as described in FIG. 4. In some embodiments, the invention provides a short component comprising a tip as described in FIG. 6.

In some embodiments, the invention provides a short component comprising a tip, wherein the tip is a vaginal dilator. In some embodiments, the invention provides a short component comprising a tip, wherein the tip is a vaginal dilator selected from Berman Vibrating Dilators, CMT Plus Size Dilators, Cool Water Cones, Soul Source Dilators, Syracuse Medical Dilators, Vaginismus Vaginal Dilators, Slimline Dilators, or Pelvic Floor Pulsator. In some embodiments, the invention provides a short component comprising a tip, wherein the tip is a Vaginismus Vaginal Dilator. In some embodiments, the invention provides a short component comprising a tip, wherein the tip is a Syracuse Medical Dilator.

In some embodiments, the invention is a single piece comprising a long component, a U-bend component, and a short component. In some embodiments, the invention is multiple pieces comprising a long component, a U-bend component, and a short component, wherein the long component is attached or affixed to the U-bend component at one end of the U-bend component and the short component is attached or affixed to the U-bend component on the other end of the U-bend component.

In some embodiments, the invention is a single piece comprising a long component, a U-bend component, and a short component, wherein the short component comprises a tip. In some embodiments, the invention is multiple pieces comprising a long component, a U-bend component, and a short component, wherein the long component is attached or affixed to the U-bend component at one end of the U-bend component, the short component is attached or affixed to the U-bend component on the other end of the U-bend component, and wherein the short component comprises a tip. In some embodiments, the tip is attached or affixed to the short component. In some embodiments, the tip can be a separate piece from the short component that can be attached or affixed to the short component.

Different embodiments may be made of many different materials. In some embodiments, one or more components can be made of medical grade plastic, non-medical grade plastic, rubber, silicone, metal, biodegradable (hydrocolloids) material, glass, acrylic, and/or combinations thereof.

In some embodiments, the invention is described in any one of FIG. 1, FIG. 2, FIG. 3, FIG. 4, FIG. 5, and/or FIG. 6. In some embodiments, the invention is as described in FIG. 1. In some embodiments, the invention is as described in FIG. 2. In some embodiments, the invention is as described in FIG. 3. In some embodiments, the invention is as described in FIG. 4. In some embodiments, the invention is as described in FIG. 5. In some embodiments, the invention is as described in FIG. 6.

FIG. 1 show a specific embodiment of the invention. 100 show a specific embodiment of a long component. 101 shows a specific embodiment of a U-bend component. 102 shows a specific embodiment of a short component. 103 shows a specific embodiment of one end of a long component. 104 shows a specific embodiment of another end of a long component attached or affixed to one end of a U-bend component. 105 shows a specific embodiment of another end of a U-bend component attached or affixed to one end of a short component. 106 shows a specific embodiment of another end of a short component. FIG. 1 shows a long component (100), a U-bend component (101), and a short component (102) wherein each component is a separate piece and the long component (100) is attached or affixed to the U-bend component (101) at one end of the U-bend component (104) and the short component (102) is attached or affixed to the U-bend component (101) on the other end of the U-bend component (105).

FIG. 2 show a specific embodiment of the invention. 200 show a specific embodiment of a long component. 201 shows a specific embodiment of a U-bend component. 202 shows a specific embodiment of a short component. FIG. 2 shows a long component (200), a U-bend component (201), and a short component (202) wherein each component is a single piece.

FIG. 3 show a specific embodiment of the invention. 300 show a specific embodiment of a long component. 301 shows a specific embodiment of a U-bend component. 302 shows a specific embodiment of a short component optionally substituted with a tip. 303 shows a specific embodiment of a tip. FIG. 3 shows a long component (300), a U-bend component (301), and a short component (302) optionally substituted with a tip (303). One having ordinary skill in the art would recognize that there are multiple ways to attach a tip (303) to a short component (302). In some embodiments, the tip (303) is a vaginal dilator.

FIG. 4 show a specific embodiment of the invention. 400 show a specific embodiment of a long component. 401 shows a specific embodiment of a U-bend component. 402 shows a specific embodiment of a short component optionally substituted with a tip. 403 shows a specific embodiment of a tip. FIG. 4 shows a long component (400), a U-bend component (401), and a short component (402) optionally substituted with a tip (403). One having ordinary skill in the art would recognize that there are multiple ways to attach a tip (403) to a short component (402). In some embodiments, the tip (403) is a spherical vaginal dilator.

FIG. 5 show a specific embodiment of the invention. 500 show a specific embodiment of a long component optionally substituted with a handle. 501 shows a specific embodiment of a U-bend component. 502 shows a specific embodiment of a short component optionally substituted with a tip. 503 shows a specific embodiment of a handle. FIG. 5 shows a long component (500) optionally substituted with a handle (503), a U-bend component (501), and a short component (502). One having ordinary skill in the art would recognize that there are multiple ways to attach a handle (503) to a long component (500).

FIG. 6 show a specific embodiment of the invention. 600 show a specific embodiment of a long component optionally substituted with a handle. 601 shows a specific embodiment of a U-bend component. 602 shows a specific embodiment of a short component optionally substituted with a tip. 603 shows a specific embodiment of a tip. 604 shows a specific embodiment of a handle. FIG. 6 shows a long component (600) optionally substituted with a handle (604), a U-bend component (601), and a short component (602) optionally substituted with a tip (603). One having ordinary skill in the art would recognize that there are multiple ways to attach a tip (603) to a short component (602). One having ordinary skill in the art would recognize that there are multiple ways to attach a handle (604) to a long component (600). In some embodiments, the tip (603) is a vaginal dilator.

In some embodiments, the invention provides a long component (for example, 100, 200, 300, 400, 500, and/or 600) wherein the long component is at least 30 cm (˜12 in.). In some embodiments, the long component (for example, 100, 200, 300, 400, 500, and/or 600) is between 30 cm (˜12 in.) and 90 cm (˜35 in.).

Methods of Treating Vaginal Pain

The device described herein can be used for therapy in reducing vaginal pain experienced during penetration. Dynamic stretching works to enhance muscle extensibility. Muscles in the vagina would adhere to these same principles. (Int. J Sports Phys Ther. 2012 February; 7(1): 109-119) and J Strength Cond Res. July 2008; 22(4): 1286-1297.) In some prescribed therapies, a patient dynamically stretches her vagina for about 5-10 seconds and repeats about 10 times and does this routine three time a day. In other prescribed therapies, a patient dynamically stretches her vagina for about 90 continuous seconds two times a day. In each prescribed therapy, about 8-10 minutes of static stretching follows and usually includes a dilator. A method of treating vaginal pain using the therapeutic tool described herein is provided.

In some embodiments, the invention provides a method of treating vaginal pain using a therapeutic tool comprising a long component, a U-bend component, and a short component wherein:

the long component is at least 30 cm (˜12 in.).

In some embodiments, the invention provides a method of treating vaginal pain using a therapeutic tool comprising a long component, a U-bend component, and a short component wherein:

the long component is at least 30 cm (˜12 in.); and

the short component is optionally attached with a tip.

In some embodiments, the invention provides a method of treating vaginal pain using a therapeutic tool comprising a long component, a U-bend component, and a short component wherein:

the long component is at least 30 cm (˜12 in.);

the short component is optionally attached with a tip; and
the tip is a vaginal dilator.

In some embodiments, the invention provides a method of treating vaginal pain using a therapeutic tool comprising a long component, a U-bend component, and a short component wherein:

the long component is between 30 cm (˜12 in.) and 90 cm (˜35 in.).

In some embodiments, the invention provides a method of treating vaginal pain using a therapeutic tool comprising a long component, a U-bend component, and a short component wherein:

the long component is between 30 cm (˜12 in.) and 90 cm (˜35 in.).; and

the short component is optionally attached with a tip.

In some embodiments, the invention provides a method of treating vaginal pain using a therapeutic tool comprising a long component, a U-bend component, and a short component wherein:

the long component is between 30 cm (˜12 in.) and 90 cm (˜35 in.);

the short component is optionally attached with a tip; and
the tip is a vaginal dilator.

In some embodiments, the invention provides a method of treating vaginal pain using a therapeutic tool comprising a long component, a U-bend component, and a short component wherein:

the long component is retractable and when extended is between 30 cm (˜12 in.) and 90 cm (˜35 in.).

In some embodiments, the invention provides a method of treating vaginal pain using a therapeutic tool comprising a long component, a U-bend component, and a short component wherein:

the long component is retractable and when extended is between 30 cm (˜12 in.) and 90 cm (˜35 in.).; and

the short component is optionally attached with a tip.

In some embodiments, the invention provides a method of treating vaginal pain using a therapeutic tool comprising a long component, a U-bend component, and a short component wherein:

the long component is retractable and when extended is between 30 cm (˜12 in.) and 90 cm (˜35 in.);

the short component is optionally attached with a tip; and
the tip is a vaginal dilator.

In some embodiments, the invention provides a method of treating a human female having vaginal pain during vaginal penetration comprising a therapeutic tool comprising a long component, a U-bend component, and a short component wherein:

the long component is between 30 cm (˜12 in.) and 90 cm (˜35 in.);

the short component is optionally attached with a tip; and
the tip is a vaginal dilator.

All literature and patent citations above are hereby expressly incorporated by reference at the location of their citation. Specifically cited sections or page of the above cited works are incorporated by reference with specificity. The invention has been described in detail sufficient to allow one of ordinary skill in the art to make and use the subject matter of the following Claims. It is apparent that certain modifications or the method and compositions of the following Claims can be made within the scope and spirit of the invention.

What is claimed is:

1. A therapeutic tool comprising a long component, a U-bend component, and a short component wherein:
the long component is at least 30 cm;
the short component is optionally attached with a tip; and
the tip is a vaginal dilator.

2. The therapeutic tool of claim 1 wherein the long component is between 30 cm and 90 cm.

3. The therapeutic tool of claim 1 wherein the long component is attached to one end of the U-bend component, and the short component is attached to the other end of the U-bend component.

4. The therapeutic tool of claim 1 wherein the vaginal dilator has a diameter ranging between 13 mm and 151 mm.

5. The therapeutic tool of claim 1 wherein the vaginal dilator has a diameter ranging between 13 mm and 44 mm.

6. The therapeutic tool of claim 1 wherein the vaginal dilator is selected from Berman Vibrating Dilators, CMT Plus Size Dilators, Cool Water Cones, Soul Source Dilators, Syracuse Medical Dilators, Vaginismus Vaginal Dilators, Slimline Dilators, or Pelvic Floor Pulsator.

7. The therapeutic tool of claim 6 wherein the vaginal dilator is a Syracuse Medical Dilator.

8. The therapeutic tool of claim 6 wherein the vaginal dilator is a Vaginismus Vaginal Dilator.

9. The therapeutic tool of claim 1 for use in treating vaginal pain.

10. The therapeutic tool of claim 9 wherein the vaginal pain is caused by Anorgasmia, Chronic Pelvic Pain, Dyspareunia, Levator Ani Spasm, Neuropathic Pain, Painful Scar Tissue, Tight Vaginal Opening, Vaginismus, or Vulvodynia.
